(19) United States
(12) Patent Application Publication
(10) Pub. No.: US 2025/0188528 A1
(43) Pub. Date: Jun. 12, 2025

(54) A POLYMERASE CHAIN REACTION (PCR) WELL INCLUDING A THERMAL CYCLING ZONE

(71) Applicant: Hewlett-Packard Development Company, L.P., Spring, TX (US)

(72) Inventors: Napoleon J. LEONI, Palo Alto, CA (US); Omer GILA, Palo Alto, CA (US)

(73) Assignee: Hewlett-Packard Development Company, L.P., Spring, TX (US)

(21) Appl. No.: 18/844,035

(22) PCT Filed: Mar. 24, 2022
(86) PCT No.: PCT/US2022/021799

§ 371 Date: Sep. 4, 2024

(51) Int. Cl. C12Q 1/6844; B01L 3/00; B01L 7/00

(52) U.S. Cl. CPC C12Q 1/6844; B01L 3/502761; B01L 7/52; B01L 2200/0668; B01L 2200/16; B01L 2300/0858; B01L 2300/1805

(57) ABSTRACT

A device includes at least one well to receive a polymerase chain reaction (PCR) mixture with the at least one well including dielectric first walls comprising a first thermal conductivity and a second wall. The second wall includes a first sheet to receive a signal causing generation of heat to form a thermal cycling zone within an interior of the at least one well in close thermal proximity to the second wall, with the first walls and the second wall defining the interior of the at least one well. A dielectric second sheet faces and is connected to the first sheet. A third sheet is connected to relative to the first sheet, with the third sheet comprising a second thermal conductivity substantially greater than the first thermal conductivity.

### BACKGROUND

Molecular diagnostics has revolutionized modern medicine. For example, molecular diagnostics have been used to better detect infectious diseases, obtain genetic information, perform pharmacogenomics, facilitate oncology, and for other purposes. Some types of molecular diagnostics may employ polymerase chain reaction (PCR) processes to rapidly make many copies of nucleic acid strands, such as RNA and/or DNA strands.

### BRIEF DESCRIPTION OF THE DRAWINGS

FIGS. 1A and 1B are each a diagram including a sectional side view schematically representing an example testing device including an example well to receive a polymerase chain reaction (PCR) mixture.

FIG. 2 is a diagram including a sectional side view schematically representing an example testing device including a plurality of example wells, each to receive a polymerase chain reaction (PCR) mixture.

FIG. 3A is a diagram including a sectional side view schematically representing an example testing device including a PCR well to receive releasable engagement from a heat block.

FIG. 3B is a diagram including a sectional side view schematically representing an example testing device including a PCR well and heat block releasably engaged relative to each other.

FIGS. 3C and 3D are each graphs schematically representing temperature changes of a PCR mixture during application of heat via example PCR wells.

FIG. 4 is a diagram including a sectional side view schematically representing an example testing device including a PCR well and heat block releasably engaged relative to each other, and a heating control system associated with the heating block.

FIG. 5A is a block diagram schematically representing an example operations engine.

FIGS. 5B and 5C are each a block diagram schematically representing an example control portion and an example user interface, respectively.

FIG. 6 is a flow diagram of an example method including performing a polymer chain reaction (PCR) via an example PCR well.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

Among other purposes, molecular diagnostics may help identify infectious diseases. One class of molecular diagnostics includes a nucleic acid amplification test, such as a polymerase chain reaction (PCR) assay to amplify target genomic material for detection. One common use of such PCR testing is for detecting viral genomic material such as, but not limited to, detecting a virus like Severe Acute Respiratory Syndrome Corona Virus 2 (SARS-CoV2), which may sometimes be referred to as COVID-19 (i.e. Corona Virus Disease of 2019).

In general terms, such nucleic acid amplification is used to make many copies of a genetic sample in order to greatly increase the accuracy and reliability of detection of the analyte of interest, such as viral genetic material. One measure of the effectiveness of such nucleic acid amplification may comprise a limit of detection, which may informally be referred to as the lowest concentration of an analyte that can be reliably detected. In general terms, the lower the limit of detection, the more accurate and reliable the test such that fewer false positives and fewer false negatives are reported.

In general terms, the PCR assay is carried out by isolating nucleic acids, such as DNA strands or RNA strands, from a sample from biological material and adding it to a PCR master mix in a well of a testing device to form a PCR mixture. Biological material may comprise human, animal, microbial, or plant biological material. In some examples, the biological material may be obtained from a human patient sample.

In many instances, because of complexity, cost, and other factors, the testing device may be located in a laboratory. However, some testing devices may be mobile and deployable in the field, such as at a point-of-care. In view of the considerable societal impact caused by some infectious diseases, providing faster and/or more mobile testing may ease the impact of such diseases by enabling early detection, tracking patterns of disease migration, treatment effectiveness, and public health decision-making.

With further reference to the actual testing, in some examples a PCR master mix, to which a sample of biological material (e.g. a biological sample) is added to form a PCR mixture, may comprise components to execute the basic steps of a polymerase chain reaction via thermal cycling within an appropriately sized test well. This combination of the PCR master mix and the biological sample (e.g. genetic sample) may be sometimes be referred to as a PCR mixture or a PCR sample volume. In some instances, a PCR sample volume may comprise about 25 to about 50 microliters and a testing device may provide for testing a group of wells arranged as a well plate or well chip. Via the testing device, a combination of the biological sample and the PCR master mix comprises the PCR sample volume, which is heated within each test well in a manner to perform the PCR assay.

Among other components, a PCR mixture may comprise a template nucleic acid sequence (e.g. DNA strands, RNA strands, portions thereof) and a PCR master mix, which may comprise buffers, dyes, cofactors, beads, primers (e.g. forward primer, reverse primer), probes, deoxyribose nucleotides (dNTPs), and/or enzyme DNA polymerase. The template nucleic acid string corresponds to the known target nucleic acid sequence to be amplified. In some examples, the enzyme DNA polymerase may comprise a Taq DNA polymerase such as a thermophilic eubacterial microorganism (Thermus aquaticus). In some examples, the cofactor may comprise Magnesium Chloride or Magnesium Sulfate. In some examples, the water may comprise nuclease-free water or PCR-grade water. In some instances, the PCR master mix also may be referred to as a PCR super mix or a PCR ready mix. Once the components appropriate for a desired type of PCR test are selected, they are added at appropriate concentrations in combination to prepare a batch mixture, which is then divided among multiple PCR wells. A volume of a PCR mixture (i.e. PCR sample volume), which includes the genetic sample and the PCR master mix per well may comprise 25 to 50 microliters (μL). At least some example PCR master mixes are available commercially from a number of sources such as, but not limited to, Sigma-Aldrich, Inc. of Saint Louis, Missouri, United States or at www.sigmaaldrich.com.

A PCR assay test involves thermal cycling including a first “denaturation” step (i.e. phase) in which the PCR sample volume (i.e. PCR mixture) is heated to about 90 degrees Celsius to about 98 degrees Celsius, which causes double-stranded nucleic acid (DNA or RNA) within the PCR mixture (i.e. PCR sample volume) to melt apart by breaking the hydrogen bonds between complementary bases, yielding two single-stranded nucleic molecules. A second step (i.e. phase) in the thermal cycling of a PCR assay test may comprise annealing in which less heat is applied to lower the reaction temperature to about 50-65° C., which allows annealing of the primers to each of the single-stranded nucleic acid templates as part of the reaction process. A third step (i.e. phase) of the thermal cycling of a PCR assay test may comprise elongation (i.e. extension) in which the heat applied to the PCR sample volume is selected to create a reaction temperature suitable for the particular nucleic acid (e.g. DNA) polymerase used. In some examples, one target activity temperature for a thermostable nucleic acid polymerase including Taq polymerase (e.g. a thermophilic eubacterial microorganism, Thermus aquaticus) is approximately 65-80° C. In this third step, the nucleic acid polymerase synthesizes a new nucleic acid strand complementary to the nucleic acid template strand by adding free nucleotide triphosphates (dNTPs) from the reaction mixture. In some examples, the temperature used in these three phases of thermal cycling may vary depending on the length of the nucleic acid strand, the time available, the type of target (e.g. RNA, DNA, etc.), the density of polymerase and primers, and/or other parameters.

Some types of implementing a PCR assay test may include combining the second and third phases (annealing and elongation) by heating the PCR sample mixture (after a denaturation phase) within a temperature range having values somewhere between the above-mentioned respective annealing and elongation temperature ranges to contemporaneously perform annealing and elongation.

In general terms, repetition of thermal cycles in performing the PCR assay test may result in an exponential increase in the quantity of the target nucleic acid sequence (e.g. DNA or RNA) to be amplified, which may sometimes be referred to the amplicon. Each cycle doubles the number of nucleic acid molecules (amplicons) amplified from the nucleic acid sequence template. For instance, in some implementations, repeating the PCR process for 30 cycles may produce on the order of 2³⁰ molecules of the target nucleic acid sequence. Of course, the number of cycles may vary depending on the amplification efficiency, detection limit, or analyte of interest, with some PCR processes utilizing thermal cycles between about 20 to about 40 cycles.

Once a sufficient number of cycles has been performed to obtain a desired quantity of the amplicons, the testing device hosting the PCR well(s) (or another testing device) may be used to detect the analyte of interest.

In some arrangements, optical detection may be used to detect output elements of the PCR assay test. This optical detection may be expressed as output element signal intensity, which may indicate a presence, a quantity, and/or a concentration of a particular analyte (e.g. virus particle, other) to which the output element is attached (e.g. bonded). In some arrangements, detection of the output elements also may be used to determine the progression of a PCR assay test.

In some arrangements, such optically-detectable output elements may comprise a fluorescent agent (e.g. dye), which may form part of the PCR master mix. One fluorescent agent may comprise a fluorophore which comprises a fluorescent chemical compound that can re-emit light upon light excitation. With this in mind, a PCR test well may comprise a wall or cover which permits the transmission of light into and/or through the PCR mixture within the test well to enable optical detection of such fluorophores to determine a presence, quantity, and/or concentration of a particular analyte.

It will be understood that other methods may be used to detect a presence, quantity, and/or concentration of the target nucleic acid (DNA or RNA) sequence of interest after amplification by the PCR process.

With this general context in mind, in at least some examples of the present disclosure, an example method and/or example device is directed to enhancing temperature control of a PCR mixture within a PCR test well by increasing a speed of thermal transfer into and/or out of the PCR test well. In some examples, the PCR test well and PCR mixture provide for a pulse-controlled amplification type of PCR test, as further described later.

In some examples of the present disclosure, a device comprises at least one well to receive a polymerase chain reaction (PCR) mixture. The at least one well may comprise dielectric first walls comprising a first thermal conductivity and a second wall. The second wall may comprise a first sheet to receive a signal causing generation of heat to form a thermal cycling zone within an interior of the at least one well in close thermal proximity to the second wall. The first walls and the second wall define the interior of the at least one well. The at least one well also comprises a dielectric second sheet and a third sheet. The second sheet faces and is connected to the first sheet. The third sheet faces and is connected to the second sheet, with the third sheet comprising a second thermal conductivity substantially greater than the first thermal conductivity.

In some examples in which the third sheet comprises a second thermal conductivity substantially greater than the first thermal conductivity of the first walls, the term substantially greater may comprise a difference of at least one of at least two orders of magnitude and at least three orders of magnitude. In some examples, with a difference of at least two orders of magnitude, a thermal conductivity of the third sheet may comprise a thermal conductivity on the order of tens (e.g. 10, 20, 30, 40) of watts per meter-Kelvin (W/m·K).

As noted above, in some examples the substantially greater difference may comprise a difference of at least three orders of magnitude. In some such examples, the thermal conductivity of the third sheet may comprise on the order of hundreds (e.g. 100, 200, 300, 400) of watts per meter-Kelvin (W/m·K). In some instances, this relationship also can be expressed as the first walls of the at least one well being made of a first class of materials comprising a thermal conductivity which is at least two orders of magnitude less than the thermal conductivity of the material forming the third sheet.

In some examples, a material of the third sheet is selected from the group of aluminum, copper, brass and combinations thereof.

In some examples, the third sheet also may comprise a thickness which is substantially greater than a thickness of the first sheet of the second wall, which may significantly enhance a mechanical stiffness of the second wall to better support the first sheet, among other features. In some examples, the third sheet comprises a thickness of about 150 microns to about 500 microns.

In some examples, the first sheet of the second wall comprises a thickness of about 10 to about 50 microns, and in some examples, the second sheet comprises a thickness of about 10 to about 200 microns.

In some examples, a control portion is to cause: the heating of, via the first sheet of the second wall and within the thermal cycling zone, a portion of the PCR mixture to exhibit a first temperature range; and via cessation of the heating via the first sheet of the second wall and via passive thermal transfer through at least the third sheet to a heat block, the portion of the PCR mixture to exhibit a second temperature range.

In some examples, after heating in the first temperature range and via passive thermal transfer through the third sheet, in less than about 100 milliseconds the portion of the PCR mixture is to exhibit the second temperature range comprising a variance of no more than about 2.5 degrees Celsius from a target temperature.

In some examples, the heat block is to exhibit the second temperature range, and the device further comprises: a temperature sensing element connected to the heat block; and a heating element connected to the heat block.

In some examples, the at least one well is to receive the PCR mixture comprising beads, wherein via gravity and/or application of magnetic force, the beads travel into and remain present within the thermal cycling zone.

Among other aspects, these example arrangements of the third sheet of the PCR well may overcome imperfections exhibited in at least some of the construction, materials, etc. of testing devices including PCR wells and related components. In at least this context, as part of performing some types of PCR tests, a heat sink may be coupled relative to a wall of a PCR well to help modulate the temperature of the PCR mixture within the PCR well. However, imperfections in materials and/or construction of the heat sink and of a wall of a PCR well may result in air gaps being introduced between the heat sink and the wall of the PCR well, which result in poor thermal contact between the heat sink and the wall of the PCR well to which the heat sink is to be releasably coupled. This poor thermal contact acts as a thermal gap between the between the heat sink and the wall of the PCR well. Moreover, some types of materials forming a wall of a PCR well (which is thermally coupled relative to the heat sink) may have a relatively low thermal conductivity, which also may effectively produce a thermal gap between the PCR well and the heat sink.

These thermal gaps, in turn, may significantly inhibit thermal transfer between the heat sink and the wall of the PCR well. Among other effects, this inhibited thermal transfer may cause a temperature of the PCR mixture within the well to rise several degrees Celsius above a temperature of the heat sink, which may disrupt the otherwise finely tuned temperatures at which the different phases of the PCR test (e.g. denaturation, annealing, elongation) are intended to occur within the PCR well. Such inhibited thermal transfer also may in turn inhibit temperatures of the PCR mixture from changing fast enough between desired temperatures at which denaturation, annealing, and elongation are intended to occur.

These multiple aspects of inhibiting thermal transfer may then affect the accuracy and/or sensitivity of testing performed via such PCR wells because the desired target temperatures of the PCR mixture (or portions thereof) cannot be achieved at all and/or at the right time, thereby decreasing the effectiveness and/or efficiency of the PCR process.

Similarly, imperfections in the materials and/or construction of a heating element of a PCR well relative to other layers of a PCR well also may contribute to the above-described thermal gaps, which inhibit desired thermal transfer, which in turn, also may contribute to the above-described deleterious effects in testing accuracy and sensitivity, etc.

With this in mind and as previously noted, in some arrangements of a two-phase PCR test the annealing and elongation may be intended to occur contemporaneously at a target temperature which fall between the annealing and elongation temperature ranges used in a three-phase PCR test. However, a shift in the actual temperature above the target temperature may cause too little annealing to occur and while a shift in the actual temperature below the target temperature may cause too little elongation to occur. These temperature shifts may result in a decrease in the degree of amplification during each cycle, thereby reducing the total quantity of amplified genetic sample (e.g. amplicon). This diminished quantity of the amplicon, in turn, may increase the limit of detection, thereby potentially reducing the accuracy and/or reliability of the particular PCR test, which may thereby increase a number of false negatives.

With this in mind, the above-described thermal gaps (e.g. poor thermal contact) resulting from the imperfections in construction and/or materials, as well as thermal gaps due to the types of materials, may at least partially cause the above-described temperature shifts above the target temperature in a two-phase PCR test because the thermal gaps inhibit the heat sink from accurately modulating the temperature of the PCR mixture and/or PCR well.

In sharp contrast, at least some examples of the present disclosure may provide for significantly increased temperature control of a PCR mixture in a PCR test at least by significantly increasing a rate of thermal transfer between a PCR well and a heat block (releasably secured to the PCR well) to more rapidly achieve a target temperature when a transition occurs between different phases of a PCR test and/or to maintain a target temperature at least because of the significantly increased correspondence (e.g. thermal correspondence) between the temperature of the heat block and the temperature of the PCR mixture in the PCR well.

In at least some examples of the present disclosure, the significantly increased temperature control may be achieved via the third sheet of a second wall of the PCR well being relatively highly thermally conductive as previously described and being relatively significantly thick to offset or counteract at least some of the above-described issues which might otherwise introduce a thermal gap between a heat block and a heating wall of a PCR well.

Via such examples of the present disclosure, upon performing a PCR test in which annealing and elongation are intended occur contemporaneously at a single temperature (which falls between the annealing and elongation temperature ranges used in a three-phase PCR test), the actual temperature may successfully be maintained at or very near the intended single temperature such that an appropriate amount of annealing and/or elongation occurs. This, in turn, results in a desired degree of amplification during each cycle, thereby producing an acceptable total quantity of amplified genetic sample (e.g. amplicon). Achieving a desired total quantity of the amplicon may, in turn, enable an acceptable limit of detection, which may increase the accuracy and/or reliability of the particular PCR test, which may thereby decrease a number of false negatives.

As previously mentioned, in some examples of the present disclosure, the PCR test may comprise a pulse-controlled amplification (PCA) type of PCR test. In a pulse-controlled amplification type of PCR test, instead of subjecting the entire volume of the PCR mixture (i.e. PCR sample volume) in the PCR well to thermal cycling, a relatively small percentage of the PCR sample volume is subjected to the thermal cycling at any given time. In some such examples of pulse-controlled amplification, heat is applied locally in a thermal cycling zone within an interior of the PCR well in close thermal proximity to a wall (e.g. second wall) of the PCR well such that just the portion of the PCR sample volume within the thermal cycling zone is subjected to the denaturation temperature of the first phase of the PCR process. In some such examples, the local heating comprises applying heat in rapid pulses (e.g. on the order of microseconds) in order to achieve the denaturation temperature of the first phase of the PCR test, with each pulse followed by a relatively rapid cooling within the thermal cycling zone Z upon the cessation of the heating pulse. In some such examples, the portion of the PCR mixture within the thermal cycling zone which is subject to a first phase (e.g. denaturation) temperature (e.g. at least 90 degrees Celsius) comprises less than about 5 percent (e.g. 4.7, 4.8, 4.9, 5.0, 5.1, 5.2) of the overall volume of the PCR mixture. As further described later, in some examples the thermal cycling zone may comprise even smaller portions of the overall volume of the PCR mixture to which a first phase (e.g. denaturation) temperature is applied.

In some examples, with the brief exception of the rapid pulse during which denaturization is to occur within the thermal cycling zone, the entire (or substantially the entire) PCR sample volume within the PCR well is generally maintained at a target single temperature at which annealing and elongation may occur contemporaneously, which is further described later in more specific examples of the present disclosure.

Via this arrangement, in some examples during an amplification portion of a PCR test, the portion of the PCR sample volume (e.g. PCR mixture) within the thermal cycling zone is subject to both the rapid pulses of the first phase temperature (e.g. denaturization) and the generally continuous second phase temperature at which both annealing and elongation are to occur. For example, immediately after each rapid pulse to achieve the first phase temperature (e.g. denaturization) within the thermal cycling zone, rapid cooling occurs within the thermal cycling zone such that the portion of the PCR mixture within the thermal cycling zone arrives at the second phase temperature at which annealing and elongation occur, as further described later. During many repetitions of thermal cycling, the temperature of the portion of the PCR mixture outside of the thermal cycling zone remains generally constant at the target second phase temperature at which annealing and elongation occur. As a result, during the amplification portion of a PCR test, annealing and elongation (e.g. at the single second phase temperature) could potentially occur anywhere within the PCR well, while solely denaturization (e.g. via a rapid heating pulse) may occur within the thermal cycling zone in close proximity to the heating first sheet of the PCR well.

Accordingly, as part of completing the PCR thermal cycling, free nucleic acid strands within the portion of the PCR well outside of the thermal cycling zone may travel back to the thermal cycling zone so that they can be amplified. In some examples, no active mixing occurs within the PCR well, such as mixing by mechanical, thermal, an induced field, or other means. In some such examples, the nucleic acid free strands tend to propagate primarily by diffusion, and therefore a majority of the annealing and elongation will happen within and around the thermal cycling zone, with all of the denaturization (e.g. at the first phase temperature) occurring within the thermal cycling zone.

Via these arrangements, the application of local heating to cause at least a first phase temperature (e.g. denaturization) via such a thermal cycling zone may dramatically reduce the time to complete a thermal cycle, which may be on the order ones of seconds of time, such as 4 or 5 seconds. Such relatively short thermal cycles may be repeated up to hundreds of times until a sufficient number of amplicons (e.g. target nucleic acid sequences) is produced to achieve a desired limit of detection for the analyte of interest.

The relatively short thermal cycling times, in turn, may dramatically reduce the time to complete the full number of thermal cycles to perform a PCR test, which may be on the order of tens of minutes such as, but not limited to, 15 or 20 minutes. This short overall completion time stands in sharp contrast to some other PCR tests, which take considerably longer to perform.

To accentuate the efficiency of such local heating, in some examples, magnetic attraction may be used to draw components within the PCR mixture in the PCR well into the thermal cycling zone. Accordingly, in some examples, the PCR well is to receive the PCR mixture, comprising beads, into the thermal cycling zone. In some such examples, via gravity and/or application of magnetic force, the beads travel into and remain present within thermal cycling zone.

In some such examples, the PCR mixture may comprise magnetic beads which are functionalized with the single-stranded nucleic acids (e.g. target nucleic acid sequence). In some examples, the functionalized beads may be paramagnetic or may be superparamagnetic, in some examples. In some examples, the thermal cycling zone Z also may comprise a location to which magnetic forces draw the functionalized beads so that the components of the PCR mixture to be subjected to the different phase temperatures within the thermal cycling zone may arrive more quickly, which in turn increases the rate of amplification of the target nucleic acid sequence. This arrangement, in turn, heightens the speed, accuracy, and reliability of the PCR test.

With these examples of pulse-controlled amplification in mind, it will be understood that the significantly increased rate of thermal transfer achievable via at least some examples of the present disclosure may significantly enhance the precision of the local heating and rapid cooling which occurs in relation to a thermal cycling zone when performing a pulsed-controlled amplification of PCR test. In particular, in some examples, such as a two-phase PCR test via pulse-controlled amplification, the significantly increased thermal correspondence between a heat block and the PCR mixture within the PCR well helps to rapidly bring the overall volume of the PCR mixture to the target single temperature (for annealing and elongation) initially when performing a PCR test and/or after the rapid denaturation pulse. This significantly increased thermal correspondence also may help maintain the target single temperature during the contemporaneous annealing and elongation during the thermal cycle.

It will be understood that at least some examples of the present disclosure directed to increasing temperature control of a PCR mixture (e.g. the portion within the thermally cycling zone) in a PCR test may be applied to a three-phase PCR test at least to the extent that significantly increasing a rate of thermal transfer between a PCR well and a heat block may enhance more rapidly achieving a target temperature in any one of the three phases of thermal cycling, such as when a transition occurs between different phases and/or maintaining a target temperature, at least because of the significantly increased correspondence (e.g. thermal correspondence) between the temperature of the heat block and the temperature of the PCR mixture in the PCR well.

In one aspect, the significantly increased temperature control, thermal transfer, etc. relating to a single PCR well also may contribute to consistency among multiple wells of a well plate or well chip, which may contribute to overall accuracy and reliability on an overall basis for the entire well plate.

With these various examples in mind, at least some further examples may comprise an example device which comprises at least one well to receive a polymerase chain reaction (PCR) mixture, wherein the at least one well includes dielectric first walls and a second wall including a heating first sheet, wherein the second wall and the first walls define the interior of the at least one well. The at least one well also includes a dielectric second sheet facing and connected to the first sheet of the second wall and a third sheet facing and connected to the second sheet. The third sheet is to receive removably secure contact from a heat block and the third sheet comprises a thermal conductivity of between about 100 and about 400 W/m·K. A signal source is connected to, and to supply a pulse control signal to cause, the first sheet of the second wall to apply heat to the PCR mixture within a thermal cycling zone in close thermal proximity to the second wall.

In some examples, after heating a first portion of the PCR mixture within the thermal cycling zone at a denaturation temperature range, via passive thermal transfer through the third sheet, the first portion of the PCR mixture exhibits a temperature in less than 100 milliseconds which varies no more than about 2.5 C from a target second temperature of about 65 degrees Celsius.

In some examples, the first walls of the at least one well are made of a first class of materials comprising a thermal conductivity which is at least three orders of magnitude less than the thermal conductivity of the third sheet.

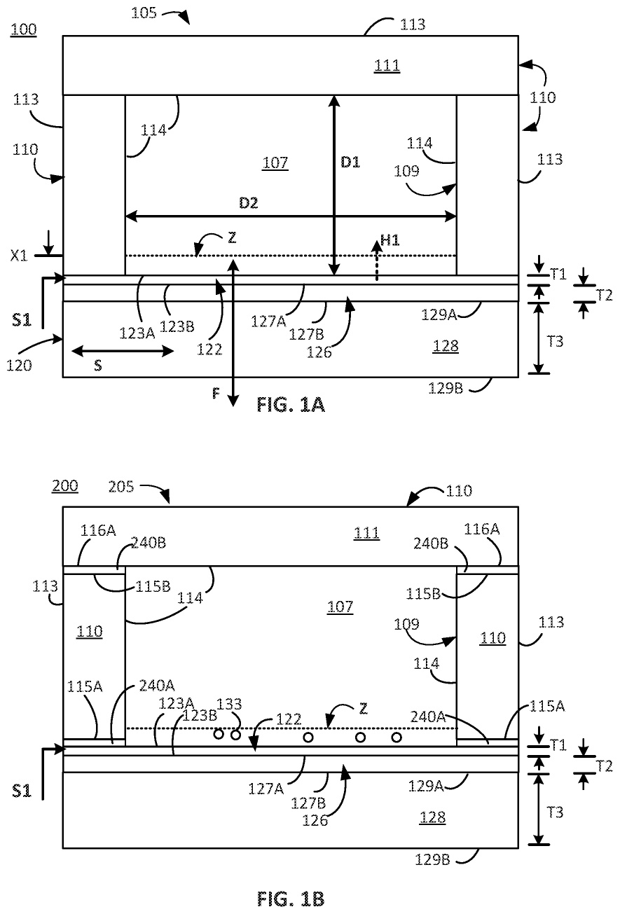

These examples, and additional examples, are described below in association with at least FIGS. 1A-6.

FIG. 1A is a side sectional view of a testing device 100 comprising an example well 105 for performing a polymerase chain reaction (PCR) test. As shown in FIG. 1A, the PCR well 105 comprises first wall(s) 110 and a second wall 120, which together define an interior 109, which in turn comprises a closed container, e.g. sealed volume. In some examples, depending on the particular construction of the testing device 100, one of the first walls 110 may sometimes be referred to as a cover, as represented by indicator 111. Each first wall 110 comprises an external surface 113 and opposite internal surface 114.

In some examples, the first walls 110 comprise a dielectric material. In some such examples, the first walls 110 may comprise a polymer material, such as (but not limited to) a cyclic olefin copolymer (COC) material. In some examples, the polymer material may comprise polyethylene, polypropylene, polycarbonate, polymethylmethacrylate (PMMA), and the like. In some examples, the above-mentioned materials of the first walls 110 may sometimes be referred to as a first class of materials, which comprises a first thermal conductivity. In some such examples, the first thermal conductivity is on the order of tenths (e.g. 0.3, 0.4, etc.) of watts per meters-Kelvin (W/m·K).

In some examples, the second wall 120 comprises first sheet 122 (e.g. layer), which comprises a first surface 123A and an opposite second surface 123B. The first surface 123A is exposed to and partially defines an interior 109 of PCR well 105, such that the first walls 110 and the first sheet 122 of the second wall 120 define the interior 109 of the PCR well 105. The interior 109 defines a receptacle to receive and then sealingly contain a polymerase chain reaction (PCR) mixture 107. In some examples, the interior 109 of the PCR well 105 may comprise a volume of about 20 to about 30 microliters, and in some examples 25 microliters. In some such examples, a volume of the PCR mixture 107 comprises approximately the same volume as the interior 109 of PCR well 105 such that no air or minimal air is present with the PCR mixture 107 within the interior 109 of the PCR well 105.

In some examples, the PCR mixture 107 may comprise at least some of substantially the same features and attributes of the previously described PCR mixtures, which comprise at least a target genetic material (e.g. target nucleic acid sequence) and a PCR master mix, with the target genetic material corresponding to the analyte of interest. The target genetic material may also sometimes referred to as an amplicon in the context of the nucleic acid amplification testing.

The first sheet 122 (of the second wall 120) may comprise an electrically activatable heating element suitable to generate heat within the PCR well 105 for performing the PCR test. In some examples, the first sheet 122 may comprise an electrically conductive material, and in some instances may be referred to as a heating foil or a heating sheet. Upon application of an electrical signal to induce heating, this electrically conductive material will generate power (P) depending on its resistivity (R) and current (I) where P=I\hat{ }2×R. Accordingly, in some instances, the first sheet 122 also may sometimes be referred to as being an electrically resistive sheet.

In some examples, the first sheet 122 may comprise a thickness (T1) of about 10 to about 50 microns, a thickness of about 15 to about 40 microns, or a thickness of about 20 to about 30 microns, and in some examples, a thickness of about 25 microns. In some examples, the first sheet 122 may comprise a material selected from the group of stainless steel, brass, titanium, tantalum, tungsten, aluminum, copper, platinum, gold, silver, zinc, indium tin oxide (ITO), and combinations thereof. In some such examples, the first sheet material is a stainless steel 304 material and in some other examples, the first sheet material is a stainless steel 306 material.

In some examples, the second wall 120 may further comprise a second sheet 126 (e.g. layer), which comprises a first surface 127A and opposite second surface 127B. In some such examples the second sheet 126 may comprise a dielectric material and which acts to secure a third sheet 128 (of the second wall 120) relative to the first sheet 122. In one aspect, the dielectric material may act to electrically isolate the first sheet 122 from the third sheet 128, which may comprise an electrically conductive, metal material in some examples.

In some examples, the second sheet 126 may comprise a thickness (T2) of about 10 microns to about 200 microns. The thickness may depend on the type of material forming the second sheet 126, as further described below.

In some examples, the second sheet 126 comprises an adhesive layer, such as a pressure sensitive adhesive (PSA) layer. In some examples the second sheet 126 may comprise a PSA layer having a thickness up to 200 microns. In some of these examples, the second sheet 126 may comprise a thickness (T2 in FIG. 1A) of about 10 to about 50 microns. In some examples, the second sheet 126 may comprise a material including both thermosetting and thermoplastic properties. In some examples, a material exhibiting thermosetting properties comprises polymer materials which are liquid or a soft solid, and which become irreversibly rigid when heated at least due to cross-linking between polymer chains, which cures the plastic. On the other hand, in some examples, a material exhibiting thermoplastic properties comprises polymer materials which soften when heated and which harden when cooled. With this in mind, in some such examples, the material of the second sheet 126 may comprise acrylic adhesive materials. In some of these examples, the second sheet 126 may comprise a thermal bonding adhesive, such as but not limited to: a Pyralux®-based material from DuPont de Nemours, Inc. of Wilmington, Delaware; and a FastelFilm material obtainable from Fastel Adhesives and Substrate Products via www.fasteladhesives.com; and the like.

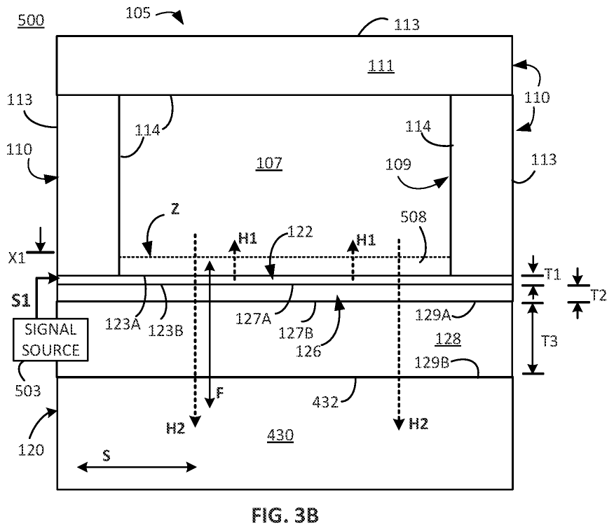

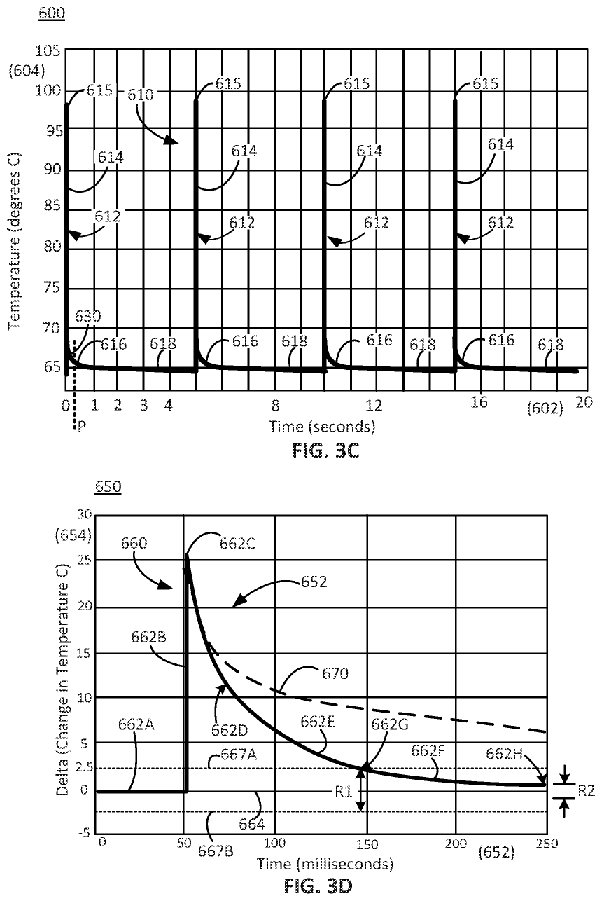

In some examples, the second wall 120 comprises the third sheet 128, which includes a first surface 129A facing and secured to the second surface 127B of the second sheet 126. The third sheet 128 also comprises an opposite second surface 129B to be releasably secured relative to a heat block (e.g. 430), as further described later in association with at least FIGS. 3A-4.

In some examples, the third sheet 128 comprises a thermally conductive metal sheet. In some examples, the third sheet 128 comprises a second thermal conductivity which is substantially greater than a first thermal conductivity of the dielectric first walls 110. In some such examples, in this context the “substantially greater” difference comprises a difference of at least two orders of magnitude difference, which in some examples corresponds to the second thermal conductivity being at least on the order of tens (e.g. 10, 20, 30, 40, etc.) of watts per meter-Kelvin (W/m·K). In some examples, the “substantially greater” difference comprises a difference of at least three orders of magnitude difference, which in some examples corresponds to the second thermal conductivity being at least on the order of hundreds (e.g. 100, 200, 300, 400, etc.) of watts per meter-Kelvin (W/m·K).

As previously explained, some examples of the present disclosure which provide a third sheet comprising a relatively high thermally conductive material may act to increase the uniformity of and/or a speed of thermal transfer into and out of the PCR mixture 107 within the PCR well 105, which may enhance the efficiency and effectiveness in performing aspects of a PCR test, which in turn may enhance the accuracy and/or reliability of results produced via such testing. At least some further aspects associated with the relatively high thermal conductivity of the third sheet 128 are further described in association with at least FIGS. 3B-3C such as, but not limited to, the speed with which a target temperature of the PCR mixture 107 may be achieved after a heating pulse to cause a denaturation phase of the PCR test is applied.

In some examples, the third sheet 128 may comprise a material selected from the group of aluminum, copper, brass, and combinations thereof or other thermally conductive materials having a thermal conductivity greater than tens of W/m·K. In some examples, the materials of aluminum, copper, or brass comprise a thermal conductivity on the order of hundreds of W/m·K. In one aspect, a copper material may comprise a thermal conductivity of about 380 W/m·K to about 400 W/m·K, such as about 385 W/m·K in some examples. In one aspect, an aluminum material may comprise a thermal conductivity of between about 235 W/m·K and about 245 W/m·K, such as about 240 W/m·K. In one aspect, a brass material may comprise a thermal conductivity of between about 110 W/m·K and about 130 W/m·K, such as 120 W/m·K.

In some examples, the third sheet 128 may comprise a thickness (T3 in FIG. 1A) of about 150 microns to about 500 microns, and in some examples a thickness of about 250 to about 400 microns. The thickness provides a mechanical stiffness sufficient to resist or prevent deformation of the first sheet 122 (e.g. heating element) of the second wall 120, which may comprise a relatively thin metal foil (e.g. 10 to 50 microns) in some examples. Accordingly, this arrangement may make the PCR well 107 more robust during physical handling, thereby helping to ensure the accuracy and reliability of PCR testing due to the enhanced physical integrity of the structure defining the PCR well, including its heating components, thermal transfer components, and/or other components. Among other forms of physical handling, upon the PCR well 105 (e.g. a consumable cartridge) becoming releasably secured relative to a heat block (e.g. 430 in FIGS. 3A-4), the significant thickness and relative rigidity of the third sheet 128 (of the PCR well 105) may help to maintain the ability of relatively high thermal transfer by helping to prevent bending, buckling, etc., which might otherwise contribute to undesired thermal gaps between the PCR well 105 and the heat block.

In one aspect, the previously-described above significant thickness and the relatively high thermal conductivity of the third sheet 128 together contribute to relatively rapid and relatively uniform thermal transfer in a first orientation (as represented by directional arrow F) between the heat block (e.g. 430 in FIG. 3A-4) and the PCR mixture 107 in the interior 109 of the PCR well 105. In one aspect, the third sheet 128 also may comprise a relative high thermal conductivity in a second orientation (as represented via directional arrow S) which is generally perpendicular to the first orientation. In some instances, the first orientation (F) may sometimes be referred to as a vertical orientation while the second orientation(S) may sometimes be referred to as a lateral orientation. This relatively high thermal conductivity in the lateral orientation may overcome imperfections in materials and/or construction (e.g. non-planar deflections) exhibited in layers of the second wall 120 of the PCR well 105, exhibited in the releasable engagement of a wall (e.g. second wall 120) of a PCR well 105 relative to a heat block (e.g. 430), and/or other imperfections which may produce air gaps, which impede thermal transfer. In particular, such imperfections may result in poor thermal contact between the layers of the second wall 120 (of the PCR well 105) and a heat block (e.g. 430 in FIGS. 3B, 4) which may inhibit desired thermal transfer in the first orientation (F) between the heat block and the second wall 120. Because such imperfections also likely extend somewhat in the second orientation along the surfaces of the second wall 120 and the heat block (e.g. 430), providing a third sheet 128 with relatively high thermal conductivity in the lateral orientation (second orientation S) may enhance faster and more uniform lateral distribution of thermal energy across or along the entire third sheet 128, thereby minimizing the impact of locations (due to imperfections) at which poor thermal transfer may be occurring, which in turn promotes thermal transfer in the first orientation (F), which is of primary interest.

With this in mind, it is further noted that the above-referenced heat block (e.g. 430 in FIG. 4) also may comprise a material having a relatively high thermal conductivity, such as Aluminum or other materials with appreciable rigidity and suitable for rapid thermal transfer. In some examples, the heat block may comprise a heat sink configuration including a plurality of fins to facilitate cooling from ambient air surrounding the heat block.

In some examples, the second sheet 126 of the second wall 120 is directly connected to the first sheet 122 and the third sheet 128 of the second wall 120 is directly connected to the second sheet 126. In some such examples, this arrangement also may sometimes be described as the second wall omitting intervening structures between the second sheet 126 and the third sheet 128 and/or omitting intervening structures between the first sheet 122 and the second sheet 126. Moreover, as previously noted the first surface 123A of the first sheet 122 (e.g. heating element) is directly exposed to and partially defines the interior 109 of the PCR well 105, such that no intervening structure/layer is present between the heating element (e.g. 122) and the PCR mixture 107 within the interior 109 of the PCR well 105.

Further details regarding heating, thermal transfer, etc. involving the second wall 120 of the PCR well 105 are further described later in association with at least FIGS. 3A-5A and 6.

In some examples, the PCR mixture 107 may comprise such PCR mixtures suitable for performing pulse-controlled amplification (PCA)-type polymerase chain reactions. Accordingly, the PCR mixture may sometimes be referred to as a PCA-PCR mixture.

As previously mentioned, it will be understood that in some examples such as some implementations of PCA-type PCR reactions, the second and third phases (annealing and extension) of thermal cycling (e.g. within zone Z) may be combined, e.g. performed contemporaneously (e.g. together) at a single target temperature which falls between about 63 degrees Celsius (e.g. 62.8, 62.9, 63, 63.1, 63.2) and about 67 degrees Celsius (e.g. 66.8, 66.9, 67, 67.1, 67.2). In some such examples, the single target temperature may comprise about 65 degrees Celsius (e.g. 64.8, 64.9, 65, 65.1, or 65.2) or may comprise about 64 degrees Celsius (e.g. 63.8, 63.9, 64, 64.1, or 64.2).

In some examples, the thermal cycle for a polymerase chain reaction (PCR), according to a pulse-controlled amplification method, may be triggered by applying a current pulse of between about 20 Volts to about 60 Volts, and having a duration of about 0.3 to about 2 milliseconds. In some such examples, the current pulse may comprise about 40 Volts with a pulse duration of about 1 millisecond. In some such examples, the current pulse may comprise on the order of 100 amps, such as 105 amps. It will be understood that the various above-identified example values of current pulse parameters may be used to achieve a target temperature rise at the surface of about 25 to about 35 degrees Celsius, which may generated by a net heat flux of about 1 to about 2.5 MWatts/m\hat{ }2 applied for about 1 milliseconds. With this in mind, it will be understood that prior to application of the first heating pulse in PCA-PCR test, in cooperation with a heat block (e.g. 430 in FIGS. 3B, 4) the first sheet 122 may apply heat to bring and maintain the PCR mixture 107 within the PRC well 105 at a target baseline temperature such as, but not limited to, the above-identified single target temperature at which annealing and elongation are to occur. Accordingly, in some such examples, the target baseline temperature may comprise about a single target temperature selected from a range between about 63 degrees Celsius and about 67 degrees Celsius. Using the examples above, in some examples the single target temperature may comprise 64 degrees Celsius or 65 degrees Celsius. With this in mind, the above-described target temperature rise of 25 to 35 degrees Celsius would bring the temperature of the PCR mixture 107 from 64 or 65 degrees Celsius to a temperature of about 90 degrees Celsius to about 98 degrees Celsius). In some examples, the temperature of the first phase (e.g. denaturation) might exceed 100 degree Celsius for a short period of time for up to a few milliseconds, in some examples. In some examples, this temperature range which is sufficient to subject a portion of the PCR volume within the thermal cycling zone Z to the desired temperature for a first phase (e.g. denaturation) in a thermal cycle of the PCA-type PCR test.

It will be understood that the above-identified parameters may vary somewhat depending on a size of the PCR well 105, volume of the PCR mixture 107, as well as the size, materials, and/or shape of the first sheet 122 (e.g. heating element) of the second wall 120 by which the heat is generated, etc. It will be understood that in some examples, the heat generated, maintained, etc. within the interior 109 of PCR well 105 also may be affected, controlled, etc. in association with a heat block, as previously described and as further described in association with at least FIGS. 3A-6.

As represented via dashed line Z in FIG. 1A, in some examples a zone in which the thermal cycling occurs may sometimes be referred to as a general thermal cycling zone which is within a predetermined distance X1 of the first surface 123A (of the first sheet 122) of the first wall 110 of the PCR well 105 through which the heat is generated and applied. In some examples, this distance X1 may correspond to, and sometimes be referred to as, being within a close thermal proximity to the first wall 110.

In some examples, the predetermined distance X1 may be expressed as a percentage of the height D1 of the interior 109 of the PCR well 105. In some examples, the percentage may comprise at least about 5 percent of the height D1, while in some other examples, the percentage may comprise at least about 6 (e.g. 5.8, 5.9, 6.0, 6.1, 6.2) percent, at least about 7 (e.g. 6.8, 6.9, 7.0, 7.1, 7.2) percent, at least about 8 (e.g. 7.8, 7.9, 8.0, 8.1, 8.2) percent, at least about 9 (e.g. 8.8, 8.9, 9.0, 9.1, 9.2) percent, or at least about 10 (9.8, 9.9, 10, 10.1, 10.2) percent.

In some of these examples in which the height D1 may comprise about 1500 microns (i.e. 1.5 millimeters) and the predetermined distance comprises about 5 percent of the height D1, then X1 would comprise about 75 microns.

Conversely, in some of these examples in which the height D1 may comprise about 300 microns (i.e. 0.3 millimeters) and the predetermined distance comprises at least about 5 percent of the height D1, then X1 would comprise about 15 microns.

In some examples, the predetermined distance X1 may be less than 5% of the height D1 of the interior 109 of the PCR well 105.

In some such examples, a width D2 of the interior 109 of the PCR well 105 may comprise about 4 millimeters, while in some other examples the width D2 may comprise between about 1 millimeter and about 10 millimeters. In some examples, the width D2 may correspond to a diameter if the well 105 has a circular cross-sectional shape or correspond to a greatest cross-sectional dimension of the well 105 comprises a non-circular cross-sectional shape.

In some examples, the general thermal cycling zone Z also may comprise a location to which magnetic forces draw superparamagnetic beads (e.g. functionalized with single-stranded nucleic acids) to heighten the effectiveness of the pulse-controlled amplification of the PCR process. One example implementation is later show in association with at least FIG. 1B.

With appreciation for the previously-described to examples of the present disclosure which introduced at least some aspects of a PCA-type, PCR test, at least some further aspects of performing such tests will be described in association with the FIGS. 1A-6, beginning with at least FIG. 1A.

Accordingly, assuming complete preparation of the PCR well 105 including the filling of PCR well 105 with the PCR mixture 107 and bringing the PCR mixture 107 to a baseline or starting target temperature, the thermal cycling to perform pulse-controlled amplification may be initiated.

With this in mind, upon receiving a signal (S1) from signal source (e.g. 503 in FIG. 3B), the first sheet 122 of second wall 120 generates heat (represented via directional arrow H1) for application to the PCR mixture 107 within PCR well 105. While FIG. 1A depicts a single directional arrow H1, it will be understood that the heat H1 may be generated and applied across and along substantially the entire first surface 123A of first sheet 122.

Moreover, via the signal S1 from signal source (503 in FIG. 3B) and the first sheet 122 (i.e. the heating sheet), heat is applied in controlled pulses in order to amplify (i.e. pulse-controlled amplification) reaction processes involving the PCR mixture 107 within the thermal cycling zone (Z) within the interior 109 of the PCR well 105. As previously noted, in some such examples, the portion of the PCR mixture 107 in the thermal cycling zone Z which is subjected, via the local heating pulses to the respective first and second temperature phases (e.g. for denaturization, annealing, elongation) comprises less than about 5 percent (e.g. 4.5, 4.6, 4.7, 4.8, 4.9, 5.0, 5.1, 5.2, 5.3, 5.4, 5.5) of the overall volume of the PCR mixture 107. In some examples, portion of the PCR mixture 107 within the thermal cycling zone Z subject to the respective first and second temperature phases (e.g. denaturization and then annealing, elongation) comprises less than about 4 percent (e.g. 3.7, 3.8, 3.9, 4.0, 4.1, 4.2), less than about 3 percent (e.g. 2.7, 2.8, 2.9, 3.0, 3.1, 3.2), less than about 2 percent (e.g. 1.7, 1.8, 1.9, 2.0, 2.1, 2.2), or less than about 1 percent (e.g. 0.7, 0.8, 0.9, 1.0, 1.1, 1.2) of the overall volume of the PCR mixture 107.

Further details regarding such heating are described below in relation to at least the first sheet 122 of second wall 120 of the PCR well 105. Further details regarding the thermal cycling via application of heat are described below in association with at least FIGS. 3A-4.

In some examples, one of the first walls 110 (e.g. 111) may comprise a material though which light may be transmitted to enable optical detection of output elements (e.g. fluorophores, etc.) resulting from the PCA-type, polymerase chain reaction. In general terms, a fluorophore may comprise a fluorescent chemical compound that can re-emit light upon light excitation. In some examples, such optical detection enables determining a presence, a quantity, or a concentration of a particular analyte (e.g. virus particle, other) to which the output element is attached (e.g. bonded). It will be understood that output elements (e.g. labels) other than fluorophores may be used in such optical detection examples.

With regard to these example dimensions, and other example dimensions throughout examples of the present disclosure, it will be understood that at least some components, spatial relationships, etc. in the Figures may be exaggerated (e.g. either made smaller or made larger) in scale for illustrative purposes, clarity, and/or simplicity.

While FIG. 1A depicts a single directional arrow H1, it will be understood that the heat H1 may be generated and applied relatively uniformly across (e.g. along) substantially the entire first surface 123A of first sheet 122. Similarly, upon instances in which a single arrow or just a couple of arrows (e.g. H1, H2, H3, etc.) are included in the FIGS to represent application of heat, thermal transfer, and the like, it will be understood that the heat (or general thermal transfer) may be applied relatively uniformly across (e.g. along) substantially the entire surface of the particular sheet, wall, etc. through which heat (or general thermal transfer) is occurring.

In some examples, the first sheet 122 may comprise a paramagnetic material or a ferromagnetic material. However, in some examples, the first sheet 122 may comprise a non-magnetic material at least to the extent that the material may be very weakly ferromagnetic or diamagnetic, and it is not intended to magnetically attract other objects such as beads to the second wall 120.

In some example, the PCR mixture 107 (and as part of the PCA-PCR process) may comprise beads which are functionalized with single-stranded nucleic acid. In some examples, the beads comprise a material and/or structure which is magnetic such that magnetic attraction of beads to the heating element 122 (i.e. first sheet) of the second wall 120 corresponds to attracting the nucleic acid strands (within the PCR mixture 107) into close thermal proximity to second wall 120. In some examples, the magnetic beads comprise paramagnetic beads and in some examples, the magnetic beads comprise superparamagnetic beads. In some such examples, the beads may be non-magnetic such that gravity alone may pull the functionalized beads toward and into the thermal cycling zone Z. One example illustration of such beads is shown later in FIG. 1B.

In one aspect relating to such examples, the thermal cycling (to perform pulse-controlled amplification of a reaction via PCR mixture) contributes to the sensitivity in testing and/or ability to detect lower quantities or concentrations of particular analytes. In addition, via such thermal transfer arrangements (e.g. the thermally conductive sheet of the second wall), examples of the present disclosure enable testing which is more sensitive and able to detect lower quantities (or concentrations) of a particular analyte of interest (e.g. virus, other).

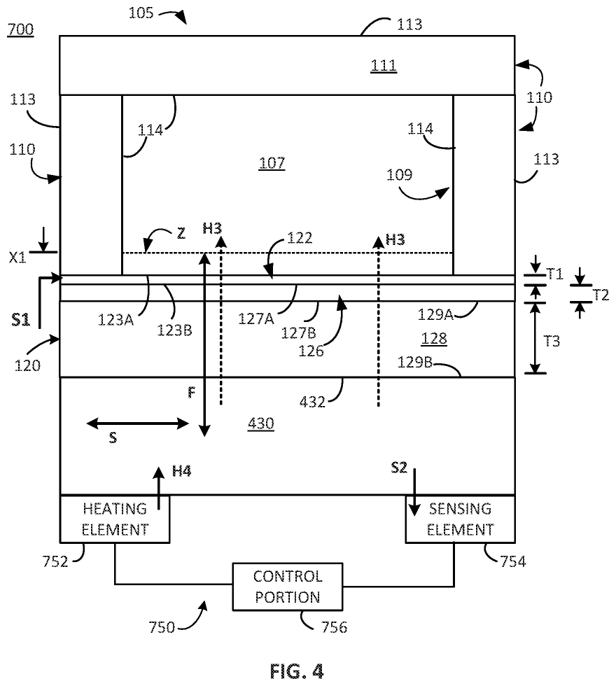

In some examples, at least some aspects of operation of, and/or monitoring of, the device 100 may be implemented via an example control portion 1000 in FIG. 5B. For instance, control portion 1000 may monitor and/or control the application of pulses from the signal source to the first sheet 122 (e.g. heating element) forming part of the second wall 120, which in turn may control at least some of the generation and application of heat within the PCR well 105.

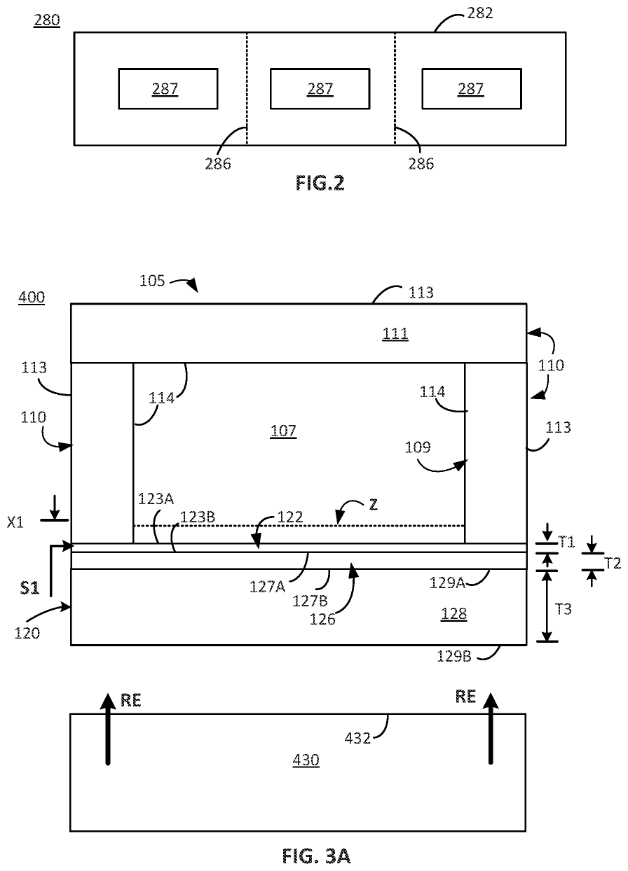

FIG. 1B is side sectional view schematically representing a testing device 200 including example PCR well 205. In some examples, the device 200 may comprise at least some of substantially the same features and attributes as the device 100 of FIG. 1A. As shown in FIG. 2, device 200 comprises a reaction well 205 comprising at least some of substantially the same features and attributes as reaction well 105 (FIG. 1A), except while further comprising adhesive layers 240A, 240B. The adhesive layer 240A is interposed between a first surface 115A of dielectric first walls 110 and a first surface 123A of first sheet 122 of second wall 120. Meanwhile, the adhesive layer 240B is interposed between a second surface 115B of dielectric first walls 110 and an inner surface 116A of the dielectric first wall 111. In some examples, the adhesive layer 240A may comprise a pressure sensitive adhesive such as, but not limited to, an acrylic-based double-sided tape, which may comprise a clear thin plastic film coated on both sides with medical grade pressure-sensitive adhesive. In some such examples, the acrylic-based double-sided tape may comprise such tapes available from Adhesives Research, Inc. of Glen Rock, Pennsylvania.

In some examples, the adhesive layer 240B may comprise a pressure sensitive adhesive such as, but not limited to, an encapsulated silicone adhesive. In some examples, the encapsulated silicone adhesive may comprise adhesive sealing film polyolefin such as, but not limited to, those available from Innovative Laboratory Products of Phoenix, Arizona. In some other examples, the adhesive layer 240B may comprise a silicon-based, adhesive-coated polyolefin film such as those available from 3M of Saint Paul, Minnesota.

As shown in FIG. 1B, in some example the PCR mixture 107 (and as part of the PCA-PCR process) may comprise beads 133 which are functionalized with a single-stranded nucleic acid, as previously noted in association with FIG. 1A. The beads 133 may comprise a material and/or structure which is magnetic such that magnetic attraction of beads to the heating element 122 of the first wall 110 corresponds to attracting the nucleic acid strands (within the PCR mixture 107) into close thermal proximity to first sheet 122 within the thermal cycling zone Z. However, in some examples, the beads may be non-magnetic such that gravity alone may pull the functionalized beads toward and into close proximity to first sheet 122 within the thermal cycling zone Z.

FIG. 2 is a side sectional view schematically representing an example testing device 280 (e.g. molecular testing device) comprising a plurality of reaction wells 287 arranged together within or on a common support structure 282. The dashed lines 286 represent a dividing line between adjacent wells 287, but such lines 286 do not necessarily correspond to a physical barrier, structure, etc. between adjacent wells 287. In some instances, the entire device may sometimes be referred to as a well plate or multi-well chip. In some examples, at least some of the wells 287 comprise at least some of substantially the same features and attributes including (or related to) the PCR well 105 in FIG. 1A and/or PCR well 205 in FIG. 1B. With further reference to FIG. 2, it will be understood that testing device 280 is not limited to the number (e.g. 3) of wells 287 shown in FIG. 2, such that device 280 may comprise a greater number or lesser number of wells 287. Moreover, in some examples, testing device 280 may comprise wells 287 arranged in a two-dimensional array (e.g. 2×2, 3×2, 4×2, etc.). In some examples, the support 282 and/or individual wells 287 may comprise a portion of, and/or be in communication with, control portion (e.g. 1000 in FIG. 5B).

In some examples, the testing device 280 may comprise additional fluidic pathways, active and/or passive fluidic control components, etc., which in turn may comprise at least a portion of (or incorporate) the control portion (e.g. 1000 in FIG. 5B). Furthermore, in some examples the testing device 280 also may be removably connectable to a console, station, or the like to support performing, monitoring, evaluating, etc. tests in the wells 287, with the respective console (or station, other) comprising at least a portion of (or incorporate) the control portion (e.g. 1000 in FIG. 5B).

As shown in the diagram of FIG. 3A, an example testing device 400 may comprise a PCR well 105 used with a heat block 430 including a first surface 432, which is brought into releasable engagement (as represented by directional arrows RE) relative to the third sheet 128 of second wall 120 of the PCR well 105. In some examples, such releasable engagement may be maintained via a clamp or other mechanical fastening arrangements.

As further shown in the diagram of a testing device 500 in FIG. 3B, with the heat block 430 releasably engaged relative to the third sheet 128 of second wall 120 of PCR well 105, a pulse-controlled amplification, PCR reaction process may be performed.

As previously explained, in some examples, prior to applying the previously-mentioned heat H1 via first sheet 122 of second wall 120, the heat block 430 is used to bring the PCR mixture 107 within the interior 109 of the PCR well 105 into a first temperature range, which is different than room temperature, such as a baseline target temperature. Further details regarding this general heating process is further described later in association with at least FIGS. 3B-4.

As previously noted, via the signal S1 applied to the first sheet 122 of the second wall 120, heat H1 is applied in controlled pulses in order to amplify (i.e. pulse-controlled amplification) reaction processes involving the polymerase chain reaction (PCR) mixture within the thermal cycling zone (Z). After the peak of each heating pulse (H1) is used to cause denaturation of the DNA strands in the PCR mixture 107 in a first phase of a thermal cycle, rapid thermal transfer occurs from the PCR mixture 107 (in PCR well 105) through the second wall 120 (including third sheet 128) into heat block 430 (as represented by dashed directional arrows H2), which significantly contributes to lowering the temperature (e.g. cooling) of the PCR mixture 107 in PCR well 105 to a narrow target temperature range which includes a single target temperature at which contemporaneous annealing and elongation are intended to occur. As previously described in introducing the examples of the present disclosure, at least the relatively highly thermally conductive material of the third sheet 128 (of the second well 120) enables the above-described rapid thermal transfer from the PCR mixture 107 to the heat block 430, which negates any significant thermal gap which might otherwise have been present in other non-example designs due to a material type and/or imperfection in construction (and/or materials) of a second wall of a PCR well.

FIG. 3C is a graph 600 including a curve 610 which schematically represents a temperature (y-axis 604) of a PCR mixture 107 within a PCR well 105 plotted over time (x-axis 602) for a series of pulse-controlled amplification thermal cycles 612. In one aspect, the curve plotted on graph 600 corresponds to thermal cycles 612 which were performed using an example device like the examples of the present disclosure described in association with at least FIGS. 1A-6 in which a third sheet 128 (FIGS. 1A-3A, 4) of a second wall 120 of a PCR well 105 comprises a relatively high thermal conductivity. The relatively high thermal conductivity may comprise a thermal conductivity which is on the order of hundreds of watts per meter-Kelvin (W/m K) (in some examples) and/or which is substantially greater than a thermal conductivity of dielectric first walls forming the PCR well. With this in mind, graph 600 provides further details regarding the heating pulses applied via the first sheet 122 of the second wall 120 of the PCR well 105 and/or regarding thermal cycling in the examples of the present disclosure.

As shown in FIG. 3C, in some examples each thermal cycle 612 has a duration of about 5 seconds, and comprises an initial pulse portion 614, cooling transition portion 616, and remainder portion 618. The initial pulse portion 614 raises the temperature from a baseline temperature of about 65 degrees Celsius to a peak 615 above 90 degrees Celsius, such as 98 degrees Celsius. Almost instantaneously after reaching peak 615, the temperature rapidly falls back to a temperature near the baseline temperature with a cooling transition portion 616 achieved via at least some examples of the present disclosure. Once the temperature has returned to a second target temperature range centered around a single target temperature such as 65 degrees Celsius, annealing and elongation occurs during the remainder portion 618 of the cycle 612 for at least the portion of the PCR mixture 107 which was subject to the denaturation temperature in the thermal cycling zone (Z in FIGS. 1A-3A, 4) in the initial pulse portion 614. These thermal cycles 612 are repeated successively for a period of time to cause the PCR reaction process to occur in a volume of the PCR mixture 107 sufficient to achieve accurate and robust testing.

As can be readily appreciated from graph 600, via the rapid thermal transfer (between the PCR well 105 and the heat block 430) enabled by the relatively high thermal conductivity of the third sheet 128 of the second wall 120 of the PCR well 105, a significant duration of each thermal cycle 612 is spent at or near the single target temperature (e.g. 65 degrees Celsius) at which annealing and elongation phases are intended to occur, as represented by remainder portion 618 of each cycle 612, until the next denaturation heating pulse portion 614 occurs. Via such arrangements, an efficiency and effectiveness of a PCR test is significantly increased at least because, after reaching the denaturation temperature, the PCR mixture 107 (e.g. portion within the thermal cycling zone Z) will have an abundance of time at the single target temperature of the second phase to enable a desired amount of, and balance between, annealing and elongation to occur.

In some examples, as can be observed from the experimentally determined data represented in FIG. 3C, a duration of the remainder portion 618 of the cycles 612 (at which annealing and elongation may occur at or near the single target temperature) may comprise at least about 95 percent (e.g. 94.6, 94.7, 94.8, 94.9, 95, 95.1, 95.2, 95.3, or 95.4 percent) of an overall duration of a thermal cycle 612, wherein the overall duration may comprise about 5 seconds in some examples. In some examples, the duration of the remainder portion 618 may comprise at least about 90 percent (e.g. 89.6, 89.7, 89.8, 89.9, 90, 90.1, 90.2, or 90.3 percent), at least about 85 percent (e.g. 84.6, 84.7, 84.8, 84.9, 85, 85.1, 85.2, 85.3, or 85.4 percent), at least about 80 percent (e.g. 79.6, 79.7, 79.8, 79.9, 80, 80.1, 80.2, or 80.3 percent), or at least about 75 percent (e.g. 74.6, 74.7, 74.8, 74.9, 75, 75.1, 75.2, 75.3, or 75.4 percent) of the overall duration of the respectively cycles 612. It will be understood that in some examples these proportions may generally be applicable to a thermal cycle 612 having a duration which is slightly more (e.g. 6 seconds) or slightly less (e.g. 4 seconds) than the duration of about 5 seconds, as depicted in FIG. 3C.

The significantly increased temperature control of the PCR well 105 as represented by FIG. 3C is further demonstrated via FIG. 3D.

FIG. 3D is a graph 650 including a curve 652 which schematically represents a temperature change (y-axis 654) of a PCR mixture 107 within the example PCR well 105 plotted over time (x-axis 602) for at least an initial pulse portion 662B (like 614 in FIG. 3C) and cooling transition 662D (like 616 in FIG. 3C) of a thermal cycle 660 (e.g. like 612 in FIG. 3C). In some such examples, the curve 652 in FIG. 3D may generally correspond to a portion of one of the thermal cycles 612 shown in FIG. 3C, such as a portion 630 of the first cycle 612 which extends between time 0 and a point in time P (as represented by the vertical dashed line in FIG. 3C) along the X-axis 602 which represents elapsed time. However, the curve 652 in FIG. 3D is plotted relative to a different quantity along the Y-axis 654, such as a change in temperature, instead of the actual temperature for a cycle 612, as represented along the Y-axis 604 in FIG. 3C. As can be appreciated from FIG. 3C, this portion 630 of a cycle 612 as represented by the curve 652 in FIG. 3D corresponds to a very short time duration of, and a small portion of, one of the thermal cycles 612 represented in FIG. 3C.

It will be understood that the curve 652 plotted in FIG. 3D comprises a composite representation of both of a numerical simulation data and observed experimental imagery/data obtained via infrared camera of the temperature behavior of the first sheet 122 within the example PCR well 105 when performing PCA-type, PCR tests. There was a high correspondence between the numerical simulation and the observed imagery/data. As previously noted, the temperature of the first sheet 122 corresponds closely with the temperature of the portion of the PCR mixture 107 within the thermal cycling zone Z. The example PRC well 105 used for such testing comprised the previously-described arrangement in which third sheet 128 of second wall 120 comprised a relatively high thermal conductivity, such as at least some examples in which the third sheet 128 was made from a copper material having a thermal conductivity of about 390 watts per meter-Kelvin (W/m·K) and a thickness of about 300 microns.

As shown in FIG. 3D, the illustrated portion of a thermal cycle 660 (generally corresponding to portion 630 in FIG. 3C) comprises a baseline portion 662A, rapid increase portion 662B, peak 662C, and cooling transition portion 662D. In some examples, the baseline portion 662A in FIG. 3D may correspond to an end segment of a remainder portion 618 of a prior heating (e.g. thermal) cycle 612 (FIG. 3C) while the rapid increase portion 662B in FIG. 3D corresponds to at least the vertically upward segment of the initial pulse portion 614 in FIG. 3C.

With further reference to FIG. 3D, the initial pulse portion 662B corresponds to heating used to nearly instantaneously achieve a denaturation temperature (e.g. at least about 90 degrees Celsius in some examples) at peak 662C (e.g. 615 in FIG. 3C), with cooling transition portion 662D (e.g. 616 in FIG. 3C) occurring generally over hundreds of milliseconds. In some examples, the peak temperature at peak 662C meets or exceeds a minimum temperature at which denaturation occurs for a portion (e.g. within the thermal cycling zone Z) of the overall volume of the PCR mixture 107, such as about 5 percent of an overall volume of the PCR mixture 107 (within a PCR well 105) or a lesser percentage, as previously described. In some examples, the cooling transition portion 662D may comprise a first cooling portion 662E which occurs within about 100 milliseconds of the rapid increase portion 662B, and then a remainder cooling portion 662F occurring thereafter until the next cycle (e.g. 612 in FIG. 3C) is to occur. A transition point 662G may mark a change from the first cooling portion 662E to the remainder cooling portion 662F. In some examples, the transition point 662G corresponds to an upper limit of a target temperature range in which annealing and/or elongation of the PCR reaction process are to occur within the interior 109 of the PCR well 105. In some examples, the target temperature range may be centered around a single target temperature, such as 65 degrees Celsius at which it is desired to cause both annealing and elongation (e.g. extension) within the PCR mixture. In some examples and as represented by the indicator R1 extending between dashed lines 667A, 667B, the target temperature range may comprise plus or minus about 2.5 degrees Celsius (e.g. 2.4, 2.45, 2.5, 2.55, 2.6) from the single target temperature of about 65 degrees Celsius (e.g. 64.8, 64.9, 65, 65.1, 65.2), such as a range of about 62.5 degrees Celsius (e.g. 62.4, 62.45, 62.5, 62.55, 62.6) to about 67.5 degrees Celsius (e.g. 67.4, 67.45, 67.5, 67.55, 67.6). In some such examples, as the portion of the PCR mixture in the thermal cycling zone (Z) cools along cooling portion 662D after the initial pulse portion 662B, the temperature of PCR mixture 107 (e.g. portion within zone Z) first reaches an upper limit (e.g. about 67.5 degrees Celsius) of this example target temperature range at transition point 662G, which is approximately 100 milliseconds after the rapid increase portion 662B of the initial pulse in thermal cycle 660. Accordingly, with these various parameters in mind, one example method employing the PCR well 105 may comprise that, after heating the first portion of the PCR mixture at the denaturation temperature range, via passive thermal transfer through the third sheet (e.g. 128 in FIGS. 1A-3A, 4), the first portion (e.g. within the thermal cycling zone Z) of the PCR mixture exhibits a temperature in less than 100 milliseconds which varies no more than about 2.5 C from a target second temperature of about 65 degrees Celsius. In some examples, this relationship also may be expressed as an example method in which, after heating in the first phase (e.g. denaturation) and via passive thermal transfer through the third sheet 128, in less than 100 milliseconds, the portion of the PCR mixture 107 enters the second phase of the thermal cycle to exhibit the second temperature range comprising a variance of no more than about 2.5 degrees Celsius from a target second temperature. As explained further in relation to at least FIG. 3D, this 100 millisecond initial cooling period 662E is dramatically faster than the cooling that might otherwise occur if a wall (e.g. second wall of the PCR well 105) were made of a material like the first walls of the PCR well.

In some examples and as represented by indicator R2 in FIG. 3D, at a point in time about 250 milliseconds from the rapid increase portion 662B, the portion of the PCR mixture 107 may further cool (via passive thermal transfer via and through third sheet 128) to exhibit a target temperature range which comprises plus or minus about 1 degrees Celsius (e.g. 0.9, 0.95, 1, 1.05, 1.1) from the single target temperature of about 65 degrees Celsius (e.g. 64.8, 64.9, 65, 65.1, 65.2), such as a range of about 64 degrees Celsius (e.g. 63.9, 63.95, 64, 64.05, 64.1) to about 66 degrees Celsius (e.g. 65.9, 65.95, 66, 66.05, 66.1). With this in mind, in some examples arrow 662H identifies an upper limit (e.g. about 66 degrees Celsius) of such example target temperature range of plus or minus 1 degree Celsius about a single target temperature of 65 degrees Celsius at about 250 milliseconds of cooling (e.g. via passive thermal transfer at least via third sheet 128) after the rapid increase portion 662B of the initial heating pulse portion 662B of a thermal cycle 660, as shown in FIG. 3D.

It is believed that in some examples, implementing a target temperature range with a variance of about 1 degree C. (e.g. plus or minus from) relative to a single target temperature (e.g. 65 degrees Celsius) (as compared to permitting a range with a variance of about 2.5 degrees Celsius) may produce significantly better pulse-controlled amplification in which both annealing and elongation are to occur contemporaneously at a single target temperature within a thermal cycle. Accordingly, one example method employing the PCR well 105 may comprise that, after heating the first portion of the PCR mixture at the denaturation temperature range, via passive thermal transfer through the third sheet (e.g. 128 in FIGS. 1A-3A, 4), the first portion of the PCR mixture exhibits a temperature, in less than 250 milliseconds, which varies no more than about 1 degree Celsius from a target second temperature of about 65 degrees Celsius.

In some examples, the target temperature range may comprise other values falling between the above-described two examples, as can be readily appreciated from the curve/graph 652 plotted in FIG. 3D between the above-described target temperature ranges at points 662G and 662H. For instance, in some examples, the target temperature range may comprise plus or minus about 2 degrees Celsius (e.g. 1.9, 1.95, 2, 2.05, 2.1) from the single target temperature of about 65 degrees Celsius (e.g. 64.8, 64.9, 65, 65.1, 65.2), such as a range of about 63 degrees Celsius (e.g. 62.9, 62.95, 63, 63.05, 63.1) to about 67 degrees Celsius (e.g. 66.9, 66.95, 67, 67.05, 67.1). In some examples, the target temperature range may comprise plus or minus about 1.5 degrees Celsius (e.g. 1.4, 1.45, 1.5, 1.55, 1.6) from the single target temperature of about 65 degrees Celsius (e.g. 64.8, 64.9, 65, 65.1, 65.2), such as a range of about 63.5 degrees Celsius (e.g. 63.4, 63.45, 63.5, 63.55, 63.6) to about 66.5 degrees Celsius (e.g. 66.4, 66.45, 66.5, 66.55, 66.6).

In some examples, a similar arrangement of example second target temperature ranges may be centered about a single target temperature other than 65 degrees Celsius to cause performance of both annealing and elongation. For example, the single target temperature may comprise 64 degrees Celsius, 64.5 degrees Celsius, 65.5 degrees Celsius, or 66 degrees Celsius.

In some examples, and as previously described, the thermal cycling zone Z within the interior 109 of the PCR well 107 exhibiting a first phase temperature (e.g. at least 90 degrees Celsius, such as at a peak 662C of a rapid increase portion 662B), may comprise less than about 5 percent, of the overall volume of the PCR mixture 107 or less than about 4 percent, less than about 3 percent, less than about 2 percent, or less than about 1 percent of the overall volume of the PCR mixture 107.

FIG. 3D also depicts a separate dashed line 670, which represents a cooling behavior of a PCR well in the absence of the relatively high thermal conductivity sheet 128 of second wall 120 (in examples of the present disclosure), such as if the second wall 120 of the PCR well was made of a material with a substantially lower thermal conductivity than a second wall including third sheet 128. It can be seen from the path of the dashed line 670 in the graph 650 of FIG. 3D that the temperature of the PCR mixture in the thermal cycling zone (Z) within the interior 109 of the PCR well 105 (particularly within the thermal cycling zone Z) would take a considerably longer time (as compared to examples of the present disclosure) to reach a target temperature range (e.g. plus or minus 2.50, 2 C, 1.50, 1 C) centered around a single target temperature of 65 Celsius (in some examples) at which annealing and elongation would be intended to occur in a second phase of a thermal cycle in a PCR test. For example, even at 250 milliseconds, the temperature represented by dashed line 670 comprises a value corresponding to about 72 degrees which has not even come close to an upper limit (e.g. 66 degrees) of a target temperature range (e.g. within 1 degree Celsius of a single target temperature of 65 degrees Celsius) for such contemporaneous annealing and elongation to get best efficiency. In such an arrangement, this relatively long period of time to reach a target temperature range (such as 65 degrees Celsius) would inhibit performance of the intended contemporaneous annealing and elongation aspects of the PCR reaction process within the PCR well 105, resulting an inadequate test within a comparable time frame and/or a test taking a significantly longer duration to complete.

With further reference to the example portion of a thermal cycle 660 in FIG. 3D and in one example of the present disclosure, the elapsed time (e.g. 100 milliseconds or 250 milliseconds) for the temperature within PCR well 105 in the thermal cycling zone (Z) to decrease from the peak temperature (at 662 C) of the initial pulse in the thermal cycle 660 to the target temperature range (e.g. 662G or 662H) is substantially less than in other arrangements lacking the relatively highly thermally conductive third sheet 128 of the second wall in examples of the present disclosure. Stated differently, via example arrangements including the relatively high thermally conductive third sheet 128 of the second wall 120, the temperature decreases (i.e. cools) from the peak 662C to a target temperature range (e.g. at 662G, at 662H) substantially less than in other arrangements with significantly less thermal conductivity within a wall of a PCR well (that releasably mates with a heat block), such as if the wall of the PCR well were made of material comparable to the material of the first walls of the PCR well.

In some such examples, the “substantially less” difference may comprise a difference of at least 4 times and in some other examples, the “substantially less” difference may comprise a difference of at least 3 times, at least 5 times, at least 6 times, at least 7 times, at least 8 times, at least 9 times, or at least 10 times.

FIG. 4 is a diagram of a testing device 700 including PCR well 105 releasably engaged relative to a heating block 430, with a heating system 750 associated with the heating block 430. In some examples, the testing device 700 may comprise one example implementation of, and/or at least some of substantially the same features and attributes of, the example testing devices described in association with at least FIGS. 1A-3D and FIGS. 5A-6.

As shown in FIG. 4, in some examples, the heating system 750 comprises a heating element 752, a temperature sensing element 754, and a control portion 756. In some examples, the temperature sensing element 754 may comprise a thermocouple, a thermistor, or an infrared sensor. In some examples, the heating element 752 is connected to and/or otherwise incorporated into the heat block 430 while in some examples, the temperature sensing element 754 is connected to and/or otherwise incorporated into the heat block 430. Via operation of control portion 756, the temperature sensing element 754 may sense a temperature of the heat block 430 (as represented by arrow S2), and using the sensed temperature, the control portion 756 may direct operation of the heating element 752 to apply heat (as represented via directional arrow H4) to maintain the heat block 430 at a target temperature. Stated differently, in some examples, the control portion 756 may cause the heat block 430 to exhibit a target temperature via controlled operation of the heating element 752 based on the sensing of, via the temperature sensing element, a temperature of the heat block.

In some such examples, via the second heating system 750, a temperature of the heating block 430 may be maintained at or near a target temperature range, such as a target temperature range at which contemporaneous annealing and elongation of PCR mixture 107 (within a thermal cycling zone Z) may be performed and/or at which a baseline temperature is established prior to application of a peak heating pulse to cause denaturation of the portion of the PCR mixture 107 within the thermal cycling zone Z. As previously noted in association with at least FIG. 3B-3D, in one aspect by maintaining the heat block 430 at a target temperature range, the arrangement significantly contributes to a rapid thermal transfer of heat out of the PCR mixture 107 (as represented via directional arrow in FIG. 3A) after a peak pulse for denaturation, and then maintaining the PCR mixture 107 within the target temperature range (e.g. within 1 degrees Celsius) centered around a single target temperature (e.g. 65 degrees Celsius) at which annealing and elongation occur, as represented by directional arrows H3 in FIG. 4.

Accordingly, in some examples, via the control portion 756 the heating system 750 may be operated to maintain, as close as possible, the heat block 430 at a single target temperature (e.g. 65 degrees Celsius) which directly corresponds to a single target temperature (e.g. 65 degrees Celsius) for contemporaneous annealing and elongation of a portion (e.g. within the thermal cycling zone Z) of the PCR mixture 107 in the PCR well 105. Among other aspects, by providing the third sheet 128 (of the second wall 120) of the PCR well 105 with the previously-described relatively high thermal conductivity, a high degree of thermal correspondence is achieved between the PCR mixture 107 (in well 105) and the heat block 430, such that the actual temperature of portion (e.g. within the thermal cycling zone) of the PCR mixture 107 in the PCR well 107 (such as depicted in FIG. 3C-3D) prior to a denaturation heating pulse (e.g. 662B in FIG. 3D) or afterwards will promptly come into equilibrium with the single target temperature being maintained at the heat block 430.

In some examples, the control portion 756 in the device 700 of FIG. 4 may comprise one example implementation of, and/or comprise at least some of substantially the same features and attributes as, the control portion 1000 in FIG. 5B.

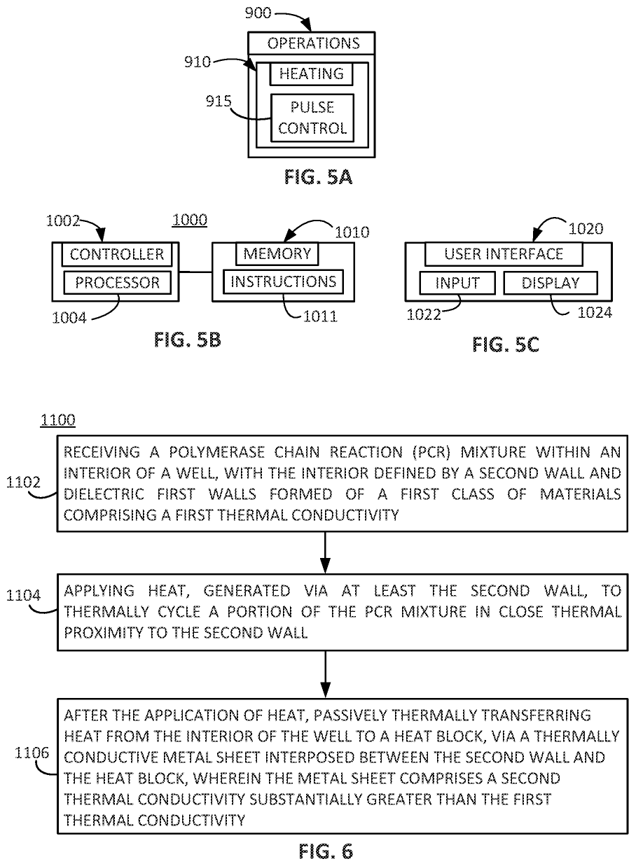

FIG. 5A is a block diagram schematically representing an example operations engine 900. In some examples, the operations engine 900 may form part of a control portion 1000, as later described in association with at least FIG. 5B, such as but not limited to comprising at least part of the instructions 1011. In some examples, the operations engine 900 may be used to implement at least some of the various example devices and/or example methods of the present disclosure as previously described in association with FIGS. 1A-4 and/or as later described in association with FIGS. 5B-6. In some examples, the operations engine 900 (FIG. 5A) and/or control portion 1000 (FIG. 5B) may form part of, and/or be in communication with, a testing device (including at least one polymerase chain reaction (PCR) well) such as the example devices and methods described in association with at least FIGS. 1A-5 and 5B-6.

In some examples and in general terms, the operations engine 900 directs, monitors, and/or reports information regarding a polymerase chain reaction (PCR) to occur within at least one well of a testing device, with the polymerase chain reaction (PCR) comprising a pulse-controlled amplification (PCA) type of polymerase chain reaction in some examples. As shown in FIG. 5A, in some examples the operations engine 900 may comprise a heating engine 910. The heating engine 910 may track and/or control heating within a PCR well, such as via applying pulses of an electric signal from signal source to a heating element (e.g. first sheet 122) forming at least a portion of a second wall (e.g. 120) of the PCR well, as described in association with at least FIGS. 1A-4. In some examples, the heating engine 910 may track and/or control heating within a PCR well, such as via managing a temperature of a heat bock (e.g. 430 in FIGS. 3A-3B, 4) relative to a temperature of a portion (e.g. within a thermal cycling zone Z) of a PCR mixture in an interior of a PCR well. In some examples and more generally speaking, the heating engine 910 may track and/or control the heating according to a pulse-controlled amplification (PCA) parameter 915 to perform the polymerase chain reaction (PCR) within the PCR well (e.g. 105) via pulse-controlled amplification.

FIG. 5B is a block diagram schematically representing an example control portion 1000. In some examples, control portion 1000 provides one example implementation of a control portion forming a part of, implementing, and/or generally managing the example testing devices (e.g. molecular testing devices), including the particular portions, components, PCR wells, signal sources, heating elements, thermally conductive sheets, heat blocks, operations, control portion, instructions, engines, functions, parameters, and/or methods, as described throughout examples of the present disclosure in association with FIGS. 1A-5A and 5C-6. In some examples, control portion 1000 includes a controller 1002 and a memory 1010. In general terms, controller 1002 of control portion 1000 comprises at least one processor 1004 and associated memories. The controller 1002 is electrically couplable to, and in communication with, memory 1010 to generate control signals to direct operation of at least some of the example molecular testing devices, including the particular portions, components, wells, signal sources, heating elements, thermally conductive sheets, heat blocks, operations, control portion, instructions, engines, functions, parameters, and/or methods, as described throughout examples of the present disclosure. In some examples, these generated control signals include, but are not limited to, employing instructions 1011 stored in memory 1010 to at least direct and manage testing operations via examples of the present disclosure. In some instances, the controller 1002 or control portion 1000 may sometimes be referred to as being programmed to perform the above-identified actions, functions, etc.

In response to or based upon commands received via a user interface (e.g. user interface 1020 in FIG. 5C) and/or via machine readable instructions, controller 1002 generates control signals as described above in accordance with at least some of the examples of the present disclosure. In some examples, controller 1002 is embodied in a general purpose computing device while in some examples, controller 1002 is incorporated into or associated with at least some of the example molecular testing devices, as well as the particular portions, components, PCR wells, signal sources, heating elements, thermally conductive sheets, heat blocks, operations, control portion, instructions, engines, functions, parameters, and/or methods, etc. as described throughout examples of the present disclosure.

For purposes of this application, in reference to the controller 1002, the term “processor” shall mean a presently developed or future developed processor (or processing resources) that executes machine readable instructions contained in a memory or that includes circuitry to perform computations. In some examples, execution of the machine readable instructions, such as those provided via memory 1010 of control portion 1000 cause the processor to perform the above-identified actions, such as operating controller 1002 to implement testing operations via the various example implementations as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 1010. The machine readable instructions may include a sequence of instructions, a processor-executable machine learning model, or the like. In some examples, memory 1010 comprises a computer readable tangible medium providing non-volatile storage of the machine readable instructions executable by a process of controller 1002. In some examples, the computer readable tangible medium may sometimes be referred to as, and/or comprise at least a portion of, a computer program product. In other examples, hard wired circuitry may be used in place of or in combination with machine readable instructions to implement the functions described. For example, controller 1002 may be embodied as part of at least one application-specific integrated circuit (ASIC), at least one field-programmable gate array (FPGA), and/or the like. In at least some examples, the controller 1002 is not limited to any specific combination of hardware circuitry and machine readable instructions, nor limited to any particular source for the machine readable instructions executed by the controller 1002.

In some examples, control portion 1000 may be entirely implemented within or by a stand-alone device.

In some examples, the control portion 1000 may be partially implemented in one of the example testing devices and partially implemented in a computing resource separate from, and independent of, the example devices but in communication with the example testing devices. For instance, in some examples control portion 1000 may be implemented via a server accessible via the cloud and/or other network pathways. In some examples, the control portion 1000 may be distributed or apportioned among multiple devices or resources such as among a server, a testing device, a user interface.

In some examples, control portion 1000 includes, and/or is in communication with, a user interface 1020 as shown in FIG. 5C. In some examples, user interface 1020 comprises a user interface or other display that provides for the simultaneous display, activation, and/or operation of at least some of the example testing devices, including the particular portions, components, PCR wells, signal sources, heating elements, thermally conductive sheets, heat blocks, operations, control portion, instructions, engines, functions, parameters, and/or methods, etc., as described in association with FIGS. 1A-5B and 6. In some examples, at least some portions or aspects of the user interface 1020 are provided via a graphical user interface (GUI), and may comprise a display 1024 and input 1022.

FIG. 6 is a flow diagram of an example method 1100. In some examples, method 1100 may be performed via at least some of the example testing devices, including the particular portions, components, PCR wells, signal sources, heating elements, thermally conductive sheets, heat blocks, operations, control portions, engines, functions, parameters, and/or methods, etc. as previously described in association with at least FIGS. 1A-5C. In some examples, method 800 may be performed via at least some testing devices, including the particular portions, components, PCR wells, signal sources, heating elements, thermally conductive sheets, heat blocks, operations, control portions, engines, functions, parameters, and/or methods, etc. other than those previously described in association with at least FIGS. 1A-5C.

As shown at 1102 in FIG. 6, in some examples method 1100 comprises receiving a polymerase chain reaction (PCR) mixture within an interior of at least one well, which is defined by a second wall and dielectric first walls. The first walls are formed of a first class of materials comprising a first thermal conductivity. As further shown at 1104 in FIG. 6, in some examples method 1800 comprises applying heat, generated via at least the second wall, to thermally cycle a portion of the PCR mixture in close thermal proximity to the second wall. As further shown at 1106 in FIG. 6, in some examples method 1800 comprises after the application of heat, passively thermally transferring heat from the interior of at least one well to a heat block, via a thermally conductive metal sheet interposed between the second wall and a heat block, wherein the metal sheet comprises a second thermal conductivity substantially greater than the first thermal conductivity.

In some examples of method 1100, passively thermally transferring heat may comprise performing the passive thermal transfer, after the heating at a denaturization temperature range, through at least the third sheet such that the first portion of the PCR mixture exhibits a first temperature in less than 100 milliseconds which varies no more than about 2.5 degrees Celsius from a target second temperature of about 65 degrees Celsius.

In some examples of method 1100, the second thermal conductivity comprises between about 100 and about 400 W/m·K.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A device comprising:
at least one well to receive a polymerase chain reaction (PCR) mixture and including:
dielectric first walls comprising a first thermal conductivity;
a second wall comprising a first sheet to receive a signal causing generation of heat to form a thermal cycling zone within an interior of the at least one well in close thermal proximity to the second wall, wherein the first walls and the second wall define the interior of the at least one well;
a dielectric second sheet facing and connected to the first sheet; and
a third sheet facing and connected to the second sheet, the third sheet comprising a second thermal conductivity substantially greater than the first thermal conductivity.

2. The device of claim 1, wherein the substantially greater thermal conductivity comprises a difference of at least one of:
at least two orders of magnitude; and
at least three orders of magnitude.

3. The device of claim 1, wherein a material of the third sheet is selected from the group of aluminum, copper, brass, and combinations thereof.

4. The device of claim 3, wherein the third sheet comprises a thickness of about 150 microns to about 500 microns.

5. The device of claim 4, wherein the first sheet of the second wall comprises a thickness of about 10 to about 50 microns, and the second sheet comprises a thickness of about 10 to about 200 microns.

6. The device of claim 1, comprising:
a control portion is to cause:
the heating of, via the first sheet of the second wall and within the thermal cycling zone, a portion of the PCR mixture to exhibit a first temperature range; and
via cessation of the heating via the first sheet of the second wall and via passive thermal transfer through at least the third sheet to a heat block, the portion of the PCR mixture to exhibit a second temperature range.

7. The device of claim 6, wherein after heating in the first temperature range and via passive thermal transfer through the third sheet, in less than 100 milliseconds the portion of the PCR mixture is to exhibit the second temperature range comprising a variance of no more than 2.5 C from a target temperature.

8. The device of claim 6, wherein the heat block is to exhibit the second temperature range, and the device further comprises:
a temperature sensing element connected to the heat block; and
a heating element connected to the heat block.

9. The device of claim 1, wherein the at least one well is to receive the PCR mixture comprising beads, wherein via gravity and/or application of magnetic force, the beads travel into and remain present within the thermal cycling zone.

10. A device comprising:
at least one well to receive a polymerase chain reaction (PCR) mixture, the at least one well including:
dielectric first walls;
a second wall including a heating first sheet, wherein the second wall and the first walls define the interior of the at least one well;
a dielectric second sheet facing and connected to the first sheet of the second wall; and
a third sheet facing and connected to the second sheet, the third sheet to receive removably secure contact from a heat block and the third sheet comprising a thermal conductivity between about 100 and about 400 W/m·K; and
a signal source connected to, and to supply a pulse control signal to cause, the first sheet of the second wall to apply heat to the PCR mixture within a thermal cycling zone in close thermal proximity to the second wall.

11. The device of claim 10, wherein after heating a first portion of the PCR mixture within the thermal cycling zone at a denaturation temperature range, via passive thermal transfer through the third sheet, the first portion of the PCR mixture exhibits a temperature in less than 100 milliseconds which varies no more than about 2.5 C from a target second temperature of about 65 degrees Celsius.

12. The device of claim 10, wherein the first walls of the at least one well are made of a first class of materials comprising a thermal conductivity which is at least three orders of magnitude less than the thermal conductivity of the third sheet.

13. A method comprising:
receiving a polymerase chain reaction (PCR) mixture within an interior of a well, with the interior defined by a second wall and dielectric first walls formed of a first class of materials comprising a first thermal conductivity;
applying heat, generated via at least the second wall, to thermally cycle a portion of the PCR mixture in close thermal proximity to the second wall; and
after the application of heat, passively thermally transferring heat from the interior of the well to a heat block, via a thermally conductive metal sheet interposed between the second wall and the heat block, wherein the metal sheet comprises a second thermal conductivity substantially greater than the first thermal conductivity.

14. The method of claim 13, wherein the passively thermally transferring heat comprises:
performing the passive thermal transfer, after the heating at a denaturation temperature range, through at least the third sheet such that the first portion of the PCR mixture exhibits a first temperature in less than 100 milliseconds which varies no more than about 2.5 degrees Celsius from a target second temperature of about 65 degrees Celsius.

15. The method of claim 13, wherein the second thermal conductivity comprises between about 100 and about 400 W/m·K.
